(19) United States
(12) Patent Application Publication
(10) Pub. No.: US 2017/0209477 A1
(43) Pub. Date: Jul. 27, 2017

(54) COMPOSITIONS FOR AMELIORATING SYSTEMIC INFLAMMATION AND METHODS FOR MAKING AND USING THEM

(71) Applicant: Vicus Therapeutics, LLC, Morristown, NJ (US)

(72) Inventors: John Maki, Mendham, NJ (US); Newell Bascomb, Hendersonville, NC (US); Timothy J. Turner, Belmont, MA (US); Fredric Young, Los Altos, CA (US)

(21) Appl. No.: 15/370,968

(22) Filed: Dec. 6, 2016

Related U.S. Application Data
(63) Continuation of application No. 14/004,828, filed on Feb. 19, 2014, now abandoned.
(60) Provisional application No. 61/452,099, filed on Mar. 12, 2011.

(51) Int. Cl. A61K 31/7072; A61K 31/192; A61K 31/551; A61K 31/5415; A61K 31/427; A61K 31/5377; A61K 31/166; A61K 38/13; A61K 31/337; A61K 31/616

(52) U.S. Cl. CPC A61K 31/7072; A61K 31/337; A61K 31/192; A61K 31/551; A61K 31/616; A61K 31/427; A61K 31/5377; A61K 31/166; A61K 38/13; A61K 31/5415

(57) ABSTRACT

In alternative embodiments the invention provides compositions, e.g., pharmaceutical compositions and preparations, formulations, kits and other products of manufacture, e.g., exemplary drug combinations packaged together or separately in blister packs, lidded blisters or blister cards, or wrapped in paper, plastic or cellophane wrappers (e.g., a shrink wrap), comprising a combination regimen of at least two active ingredients designed to diminish systemic inflammation by targeting (inhibiting) two different, but convergent, signaling pathways, i.e., the sympathetic nervous system and the lipid-derived autacoid system; and methods for making and using these compositions. In alternative embodiments, the compositions of the invention (e.g., the combination of drugs) are used to ameliorate, diminish, treat, block or prevent an inflammatory response secondary to an infection, e.g., a viral infection and/or a reactivation.

### RELATED APPLICATIONS

This U.S. utility patent application is a continuation of U.S. patent application Ser. No. 14/004,828, filed Feb. 19, 2014, now pending, which a national phase application claiming benefit of priority under 35 U.S.C. §371 to Patent Convention Treaty (PCT) International Application Serial No: PCT/US2012/028312, filed Mar. 8, 2012, which claims benefit of priority to U.S. Provisional Patent Application Ser. No. 61/452,099, filed Mar. 12, 2011, which is expressly incorporated by reference herein in its entirety for all purposes.

### FIELD OF THE INVENTION

This invention relates generally to medicine, infectious diseases and pharmaceutical formulations. In alternative embodiments, the invention provides compositions, e.g., pharmaceutical compositions and preparations, formulations, kits and other products of manufacture, e.g., exemplary drug combinations packaged together or separately in blister packs, lidded blisters or blister cards, or wrapped in paper, plastic or cellophane wrappers (e.g., a shrink wrap), comprising a combination regimen of at least two active ingredients designed to diminish systemic inflammation by targeting (inhibiting) two different, but convergent, signaling pathways, i.e., the sympathetic nervous system and the lipid-derived autacoid system; and methods for making and using these compositions. In alternative embodiments, the compositions of the invention (e.g., the combination of drugs) are used to ameliorate, diminish, treat, block or prevent an inflammatory response secondary to an infection, e.g., a viral infection and/or a reactivation.

### BACKGROUND

Systemic inflammation acts as a co-morbidity factor in certain viral infections, such as human herpes virus-8 (HHV-8) or Kaposi's Sarcoma Herpes Virus. This virus is associated with a hyperproliferative skin disorder called Kaposi's Sarcoma that is most often observed secondary to HIV-induced immunosuppression, and a rare B-cell disorder known as Castleman's Disease, and specifically the form that is more refractory to treatment known as Multicentric Castleman's Disease (MCD).

### SUMMARY

In alternative embodiments, the invention provides products of manufacture comprising a compound, a pharmaceutical composition or a formulation, a blister package, a lidded blister or a blister card or packet, a clamshell, a tray or a shrink wrap, or a kit, comprising:

(a) (i) a compound, pharmaceutical composition or formulation comprising an inhibitor of the sympathetic nervous system; and
(ii) a compound, pharmaceutical composition or formulation comprising an inhibitor of the lipid-derived autacoid system;

(b) the product of manufacture of (a), wherein the inhibitor of the sympathetic nervous system comprises a beta adrenoceptor antagonist compound or drug;

(c) the product of manufacture of (a), wherein the inhibitor of the lipid-derived autacoid system comprises a non-selective or selective COX-2 inhibiting non-steroidal anti-inflammatory compound or drug;

(d) the product of manufacture of (b), wherein the beta adrenoceptor antagonist comprises:

a propranolol, or a 2-Propanol, 1-[(1-methylethyl)amino]-3-(1-naphthalenyloxy)-, hydrochloride, or equivalent (optionally INDERAL™, AVLOCARDYL™, AVLOCARDYL RETARD™, DERALIN™, DOCITON™, INDERALICI™, INNOPRAN XL™, SUMIAL™, or ANAPRILINUM™) optionally administered at between about 10 mg up to 800 mg daily, optionally in divided doses,

a nadolol (optionally CORGARD™, ANABET™, SOLGOL™, CORZIDE™, ALTI-NADOLOL™, APO-NADOL™, or NOVO-NADOLOL™) optionally administered at between about 1 mg up to 400 mg once daily,

a timolol or timolol maleate (optionally administered at between about 1 mg up to 400 mg daily, optionally in divided doses),

a pindolol (optionally VISKEN™, BETAPINDOL™, BLOCKIN L™, BLOCKLIN L™, CALVISKEN™, CARDILATE™, DECRETEN™, DURAPINDOL™, GLAUCO-VISKEN™, PECTOBLOC™, PINBETOL™, PRINDOLOL™, or PYNASTIN™) optionally administered at between about 1 mg up to 200 mg daily, optionally in divided doses),

a labetalol (optionally NORMODYNE™, TRANDATE™, or NORMOZYDE™) optionally administered at between about 10 mg up to 4000 mg daily, optionally in divided doses),

a beta-1-selective drug,

a metoprolol (optionally administered at between about 10 mg up to 800 mg daily, optionally in divided doses),

an atenolol (optionally TENORMIN™) optionally administered at between about 1 mg up to 200 mg daily),

an acebutolol (optionally SECTRAL™, or PRENT™) optionally administered at between about 10 mg up to 2400 mg daily, optionally in divided doses),

an alpha-beta non-selective agent,

a carvedilol (optionally COREOG™, DILATREND™, EUCARDIC™, CARLOC™, CIPLA™, or COREG CR™) optionally administered at between about 1 mg up to 400 mg daily, optionally in divided doses), or

any combination thereof (e.g., comprising at least one atenolol, nadolol, metoprolol, propranolol, carteolol, carvedolol, labetalol, oxprenolol, penbutolol, pindolol, sotalol, timolol or a combination thereof);

(e) the product of manufacture of (c), wherein the non-selective or selective COX-2 inhibiting non-steroidal anti-inflammatory drug comprises:

a diaryl furanone (optionally a rofecoxib, optionally VIOXX™, CEOXX™, or CEEOXX™, optionally administered at between about 1 mg to 100 mg daily),

a diaryl pyrazole (optionally a celecoxib, optionally COBIX™, CELCOXX™, or SELECAP™) optionally administered at between about 1 mg to 800 mg daily),

an indole acetate (optionally a etodolac, optionally LODINE SR™, or ECCOXOLAC™, optionally administered at between about 10 mg to 2000 mg daily, optionally in divided doses),

a sulfonamide (optionally a nimensulide, optionally AULIN™, MESULIDE™, or NIMED™) optionally administered at between about 10 mg to 1000 mg daily),

a salicylate (optionally a acetyl salicylic acid or aspirin, optionally administered at between about 40 mg to 4000 mg daily, optionally in divided doses),

an indole or an indene acetic acid (optionally an indomethacin, optionally INDOCIN™, INDOCID™, INDOCHRON E-R™, or INDOCIN-S™, optionally administered at between about 10 mg to 400 mg daily, optionally in divided doses),

a heteroaryl acetic acid (optionally a diclofenac, optionally CATAFLAM™, or ZIPSOR™), optionally administered at between about 10 mg to 400 mg daily, optionally in divided doses),

an arylpropionic acid, optionally an ibuprofen (optionally BRUFEN™, NUROFEN™, ADVIL™, or NUPRIN™), optionally administered at between about 10 mg to 4000 mg daily, optionally in divided doses;

a naproxen, optionally ALEVE™, ANAPROX™, ANTALGIN™, FEMINAX ULTRA™, FLANAX™, INZA™, MIDOL EXTENDED RELIEF™, MIRANAXV, NALGESIN™, NAPOSIN™, NAPRELAN™, NAPROGESIC™, NAPROSYN™, NAROCIN™, PROXEN™, SYNFLEX™, or XENOBID™, optionally at 10 mg to 4000 mg daily, optionally in divided doses,

a fenamate, optionally a mefanamic acid, optionally PONSTEL™, optionally administered at between about 100 mg to 4000 mg daily, optionally in divided doses, an enolate (optionally a meloxicam, optionally MOVALISU™, MELOX™, RECOXA™, MOBIC™, MOBEC™, MOBICOX™, TENARON™, ILACOX™, MAVICAM™, MELOCAM™, or ARTRIFLAM™) optionally administered at between about 1 mg to 100 mg daily; optionally administered at between about piroxicam at 1 mg to 100 mg daily),

an alkanone (optionally a nabumetone, optionally RELAFEN™, RELIFEX™, or GAMBARAN™) optionally administered at between about 10 mg to 4000 mg daily, optionally in divided doses), or

any combination thereof (e.g., optionally comprising any non-steroidal anti-inflammatory drug (NSAID), e.g., comprising aspirin, diclofenac; diflunisal, etodolac, fenoprofen, flurbiprofen, ibuprofen, indomethacin, ketoprofen; ketorolac, meclofenamate, mefenamic acid, meloxicam, nabumetone, naproxen, oxaprozin, piroxicam, salsalate, sulindac, tolmetin, a COX-2 inhibitor (e.g., a COX-2-selective inhibitor) or a combination thereof, wherein optionally the COX-2-selective inhibitor comprises celecoxib rofecoxib, etoricoxib, valdecoxib, parecoxib, meloxicam or lumiracoxib);

(f) the product of manufacture of any of (a) to (e), wherein the compound, composition or formulation comprises or is formulated as:

a tablet, a pill, a lozenge, a capsule, a gel, a geltab, a nanosuspension, a nanoparticle, a microgel and/or a pellet, and optionally the tablet, pill, lozenge, capsule, gel, geltab, nanosuspension, nanoparticle, microgel and/or a pellet are released upon opening of a single package or packet package, or upon opening of a plurality of packages in a blister pack or in a plurality of blister packettes, or

an ampoule, a gel, a lotion, a cream, an emollient, a skin patch or adhesive, aerosol or a spray for topical application, wherein optionally ampoule, a gel, a lotion, a cream, an emollient, a skin patch or adhesive, aerosol or a spray for topical application, and optionally packaged in its own (is contained in a single (one)) tube, ampoule or packette;

(g) the product of manufacture of any of (a) to (f), further comprising instructions for use;

(h) the product of manufacture of (g), wherein the instructions for using the product of manufacture comprise instructions for use to ameliorate, diminish, treat, block or prevent a systemic inflammation, or an inflammatory response secondary to an infection, a viral infection and/or a reactivation;

(i) the product of manufacture of (g) or (h), wherein the instructions for using the product of manufacture comprise instructions for use to ameliorate, diminish, treat, block or prevent an inflammation associated with a herpes virus infection, a human herpes virus-8 (HHV-8) infection, or a Kaposi's Sarcoma Herpes Virus infection; or

(j) the product of manufacture of (g) or (h), wherein the instructions for using the product of manufacture comprise instructions for use to ameliorate, diminish, treat, block or prevent an inflammation associated with a hyperproliferative skin disorder, Kaposi's Sarcoma, an inflammation secondary to HIV-induced immunosuppression, a B-cell disorder, Castleman's Disease, or Multicentric Castleman's Disease (MCD).

In alternative embodiments, the products of manufacture of the inventions further comprise:

(a) a compound, a pharmaceutical composition or a formulation or therapy comprising:

an anti-viral or an anti-retroviral agent or therapeutic,

a nucleoside reverse transcriptase inhibitor (optionally zidovudine, optionally administered at between about 100 mg to 4000 mg daily, optionally in divided doses,

a lamivudine, optionally administered at between about 100 mg to 600 mg daily, optionally in divided doses),

a non-nucleoside reverse transcriptase inhibitor (optionally nevirapine, optionally administered at between about 10 mg to 2000 mg daily, optionally in divided doses; or an efavirenz, optionally administered at between about 10 mg to 2400 mg daily, optionally in divided doses),

a protease inhibitor,

an indinavir (optionally CRIXIVAN™), optionally administered at between about 100 mg to 4000 mg daily, optionally in divided doses,

a ritonavir (optionally NORVIR™) optionally administered at between about 100 mg to 2400 mg daily, optionally in divided doses,

an antiherpesvirus agent, or an antiherpesvirus agent capable of blocking viral replication and shedding of human herpes virus, wherein the herpesvirus is HHV-1 (Herpes Simplex Virus, or HSV 1), HHV-2 (Herpes Simplex Virus-2 or HVS2), HHV-3 (Varicella Zoster), HHV-4 (Epstein-Barr Virus, EBV), HHV-5 (cytomegalovirus, CMV), HHV-6 (roseolovirus, or herpes lymphotrophic virus), HHV-7 (roseolovirus), HHV-8 (Human Herpes Virus-8, also known as Kaposi's Sarcoma Herpes Virus, “KSHV”),

an ganciclovir (optionally CYTOVENE™; optionally administered at between about 1 mg to 4500 mg daily, optionally in divided doses) and its prodrug valganciclovir (optionally VALCYTE™; optionally administered at between about 100 mg to 4500 mg daily, optionally in divided doses),

an acyclovir (optionally ZOVIRAX™; optionally administered at between about 100 mg to 8000 mg daily, in divided doses) and its prodrug valacyclovir (optionally VALTREX™, optionally administered at between about 1 g to 10 g per day, optionally in divided doses),

an cidofovir (optionally VISTIDE™; optionally administered at between about 1 mg/kg to 400 mg/kg daily, optionally by intravenous infusion),

a famciclovir (optionally FAMVIR™; optionally administered at between about 10 mg to 4000 mg daily, optionally in divided doses),

a penciclovir (optionally DENAVIR™; optionally administered at between about 10 mg to 400 mg daily, optionally in divided doses),

a foscarnet (optionally FOSCAVIR™; optionally administered at between about 10 mg/kg to 400 mg/kg daily, optionally by intravenous infusion),

an imiquimod (optionally ALDARA™; optionally administered at between about 10 mg to 400 mg daily, optionally in divided doses),

a ribavirin (optionally REBETOL™, optionally administered at between about 1 μg/kg/wk up to 10 μg/kg/wk, optionally given by subcutaneous injection),

an interferon-alpha (optionally ROFERON™; optionally administered at between about 1 MIU (about 20 ng) to 30 MIU, optionally weekly, optionally at approximately 600 ng weekly, optionally given in divided doses by subcutaneous injection, optionally comprising its pegylated derivatives, optionally PEG-INTRON™; optionally administered at between about 1 μg/kg up to 100 μg/kg weekly, optionally given by subcutaneous injection, or

any combination thereof;

(b) a compound, a pharmaceutical composition or a formulation or therapy comprising:

a compound, a pharmaceutical composition or a formulation or therapy targeting a cell infected with human herpes virus-8 (HHV-8),

a rituximab (optionally RITUXAN™, or MABTHERA™) or other antibodies that target a CD20 antigen expressed on a B-cell, optionally administered at between about 10 mg/m² to 1000 mg/m² given by infusion, up to every two weeks),

an etoposide (optionally EPOSIN™, ETOPOPHOS™, VEPESID™, or VP-16™) or other inhibitors of a eukaryotic topoisomerase II (optionally based on podophyllotoxin), optionally administered at between about 10 mg/m² up to 100 mg/m², optionally taken orally in divided doses or given by intravenous infusion or any other route, or

any combination thereof;

(c) a compound, a pharmaceutical composition or a formulation or therapy comprising:

a compound, a pharmaceutical composition or a formulation or drug that modulates the immune system, or causes an HHV-8 reactivation due to an alteration in an immune system function,

a thalidomide, a lenalidomide, a pomalidomide, or a congener or analog (optionally THALOMID™, or REVLIMID™) optionally administered at between about 10 mg to 1000 mg daily, in divided doses, with or without concomitant glucocorticoid therapy,

a lenalidomide (optionally administered at between about 1 mg to 100 mg daily, with or without concomitant glucocorticoid therapy),

a pomalidomide (optionally administered at between about 0.1 mg to 40 mg daily, or every other day, with or without concomitant glucocorticoid therapy), or

any combination thereof;

(d) a compound, a pharmaceutical composition or a formulation or therapy comprising:

a compound, a pharmaceutical composition or a formulation or drug that modulates an immune system by interfering with a calcineurin/NFAT signaling in a T cells;

a cyclosporine, a cyclosporine A, or ciclosporin (optionally administered at between about 0.1 mg/kg/da to 20 mg/kg/da),

a tacrolimus (optionally FK-506™ or FUJIMYCIN™) optionally administered at between about 1 μg/kg/da up to 100 μg/kg/da by, optionally intravenous infusion),

a pimecrolimus (optionally ELIDEL™) (optionally administered at between about 1 μg/kg/da up to 100 μg/kg/da, optionally by intravenous infusion), or

any combination thereof,

(e) a compound, a pharmaceutical composition or a formulation or therapy comprising:

a compound, a pharmaceutical composition or a formulation or drug that acts directly or indirectly as an anti-proliferative agent for a T cell,

a sirolimus or rapamycin (optionally RAPAMUNE™) optionally administered at between about 1 mg up to 100 mg/da, optionally by oral or intravenous route,

an inhibitor of a mammalian target of rapamycin (mTORs),

an azathioprine (optionally administered at between about 0.1 mg/kg/da up to 10 mg/kg/da, optionally by oral, intravenous, or other route).

a mycophenolate mofetil or a mycophenolic acid (optionally CELLCEPT™ or MYFORTIC™) optionally administered at between about 100 mg up to 10 g/da, optionally by oral or intravenous, or

any combination thereof; or

(f) a compound, a pharmaceutical composition or a formulation or therapy comprising:

a compound, a pharmaceutical composition or a formulation or drug that interferes with signaling through an IL-6 cytokine pathway, or attenuates an immunomodulatory response induced by IL-6,

a monoclonal antibody that binds to an IL-6 receptor,

a tocilizumab (optionally ACTEMRA™ or ROACTEMRA™) optionally administered at between about 1 mg/kg up to 20 mg/kg, optionally given by intravenous infusion, optionally as often as every 28 days; optionally a dosing schedule including daily infusions,

a monoclonal antibody that adsorbs an IL-6 peptide,

an elsilimomab (optionally B-E8™) administered at between about 1 mg/kg up to 10 mg/kg every 2 weeks), optionally given with or without concomitant glucocorticoid therapy, or

any combination thereof; or

(g) a compound, a pharmaceutical composition or a formulation or therapy comprising:

a compound, a pharmaceutical composition or a formulation or drug that is a cytotoxic drug,

a compound, a pharmaceutical composition or a formulation or drug that is a cancer chemotherapeutic,

a compound, a pharmaceutical composition or a formulation or drug that induces myelosuppression,

a compound, a pharmaceutical composition or a formulation or drug that is disrupts microtubule function,

a taxane (optionally paclitaxel or TAXOL™, or docetaxel or TAXOTERE™),

a polyether macrolide (optionally halichondrin B, or eribulin or HALAVEN™),

a compound, a pharmaceutical composition or a formulation or drug that inhibits a DNA methyltransferase activity,

an azacytidine, optionally a 5-azacytidine, optionally VIDAZA™, optionally administered at between about 10 mg/m²/da up to 300 mg/m²/da, optionally by subcutaneous injection, or optionally by intravenous infusion,

a decitabine, optionally a DACOGEN™, optionally administered at between about 1 mg/m² up to 100 mg/m² up to three times daily, optionally by intravenous infusion,

a depsipeptide drug that inhibits a histone deacetylase,

a romidepsin, optionally ISTODAX™, optionally administered at between about at 1 mg/rn² up to 100 mg/m² weekly, optionally more or less frequently by intravenous infusion, or

any combination thereof.

In alternative embodiments, the invention provides methods for ameliorating, diminishing, treating, blocking or preventing a systemic inflammation, or an inflammatory response, or an inflammatory response secondary to a disease, condition, contamination, poisoning, or infection, or a viral infection and/or a reactivation, comprising:

(a) administering to an individual, a patient or an animal in need thereof a product of manufacture, a pharmaceutical composition or a formulation, a blister package, a lidded blister or a blister card or packet, a clamshell, a tray or a shrink wrap, or a kit, of claim 1 or claim 2;

(b) administering to an individual, a patient or an animal in need thereof
(i) a compound, pharmaceutical composition or formulation comprising an inhibitor of the sympathetic nervous system; and
(ii) a compound, pharmaceutical composition or formulation comprising an inhibitor of the lipid-derived autacoid system;

(c) the method of (b), wherein the inhibitor of the sympathetic nervous system comprises a beta adrenoceptor antagonist compound or drug;

(d) the method of (b), wherein the inhibitor of the lipid-derived autacoid system comprises a non-selective or selective COX-2 inhibiting non-steroidal anti-inflammatory compound or drug;

(e) the method of (c), wherein the beta adrenoceptor antagonist comprises:

a propranolol, or a 2-Propanol, 1-[(1-methylethyl)amino]-3-(1-naphthalenyloxy)-, hydrochloride, or equivalent (optionally INDERAL™, AVLOCARDYL™, AVLOCARDYL RETARD™, DERALIN™, DOCITON™, INDERALICI™, INNOPRAN XL™, SUMIAL™, or ANAPRILINUM™) optionally administered at between about 10 mg up to 800 mg daily, optionally in divided doses,

a nadolol (optionally CORGARD™, ANABET™, SOLGOL™, CORZIDE™, ALTI-NADOLOL™, APO-NADOL™, or NOVO-NADOLOL™) optionally administered at between about 1 mg up to 400 mg once daily,

a timolol or timolol maleate (optionally administered at between about 1 mg up to 400 mg daily, optionally in divided doses),

a pindolol (optionally VISKEN™, BETAPINDOL™, BLOCKIN L™, BLOCKLIN L™, CALVISKEN™, CARDILATE™, DECRETEN™, DURAPINDOL™, GLAUCO-VISKEN™, PECTOBLOC™, PINBETOL™, PRINDOLOL™, or PYNASTIN™) optionally administered at between about 1 mg up to 200 mg daily, optionally in divided doses),

a labetalol (optionally NORMODYNE™, TRANDATE™, or NORMOZYDE™) optionally administered at between about 10 mg up to 4000 mg daily, optionally in divided doses),

a beta-1-selective drug,

a metoprolol (optionally administered at between about 10 mg up to 800 mg daily, optionally in divided doses),

an atenolol (optionally TENORMIN™) optionally administered at between about 1 mg up to 200 mg daily),

an acebutolol (optionally SECTRAL™, or PRENT™) optionally administered at between about 10 mg up to 2400 mg daily, optionally in divided doses),

an alpha-beta non-selective agent,

a carvedilol (optionally COREG™, DILATREND™, EUCARDIC™, CARLOC™, CIPLA™, or COREG CR™) optionally administered at between about 1 mg up to 400 mg daily, optionally in divided doses), or

any combination thereof (e.g., comprising at least one atenolol, nadolol, metoprolol, propranolol, carteolol, carvedolol, labetalol, oxprenolol, penbutolol, pindolol, sotalol, timolol or a combination thereof); or

(f) the method of (d), wherein the non-selective or selective COX-2 inhibiting non-steroidal anti-inflammatory drug comprises:

a diaryl furanone (optionally a rofecoxib, optionally VIOXX™, CEOXX™, or CEEOXX™, optionally administered at between about 1 mg to 100 mg daily), a diaryl pyrazole (optionally a celecoxib, optionally COBIX™, CELCOXX™, or SELECAP™) optionally administered at between about 1 mg to 800 mg daily),

an indole acetate (optionally a etodolac, optionally LODINE SR™, or ECCOXOLAC™, optionally administered at between about 10 mg to 2000 mg daily, optionally in divided doses),

a sulfonamide (optionally a nimensulide, optionally AULIN™, MESULIDE™, or NIMED™) optionally administered at between about 10 mg to 1000 mg daily),

a salicylate (optionally a acetyl salicylic acid or aspirin, optionally administered at between about 40 mg to 4000 mg daily, optionally in divided doses),

an indole or an indene acetic acid (optionally an indomethacin, optionally INDOCIN™, INDOCID™, INDOCHRON E-R™, or INDOCIN-ST™, optionally administered at between about 10 mg to 400 mg daily, optionally in divided doses),

a heteroaryl acetic acid (optionally a diclofenac, optionally CATAFLAM™, or ZIPSOR™), optionally administered at between about 10 mg to 400 mg daily, optionally in divided doses),

an arylpropionic acid, optionally an ibuprofen (optionally BRUFEN™, NUROFEN™, ADVIL™, or NUPRIN™), optionally administered at between about 10 mg to 4000 mg daily, optionally in divided doses;

a naproxen, optionally ALEVE™, ANAPROX™, ANTALGIN™, FEMINAX ULTRA™, FLANAX™, INZA™, MIDOL EXTENDED RELIEF™, MIRANAXV, NALGESIN™, NAPOSIN™, NAPRELAN™, NAPROGESIC™, NAPROSYN™, NAROCIN™, PROXEN™, SYNFLEX™, or XENOBID™, optionally at 10 mg to 4000 mg daily, optionally in divided doses,

a fenamate, optionally a mefanamic acid, optionally PONSTEL™, optionally administered at between about 100 mg to 4000 mg daily, optionally in divided doses,

an enolate (optionally a meloxicam, optionally MOVALIS™, MELOX™, RECOXA™, MOBIC™, MOBEC™, MOBICOX™, TENARON™, ILACOX™, MAVICAM™, MELOCAM™, or ARTRIFLAM™) optionally administered at between about 1 mg to 100 mg daily; optionally administered at between about piroxicam at 1 mg to 100 mg daily),

an alkanone (optionally a nabumetone, optionally RELAFEN™, RELIFEX™, or GAMBARAN™) optionally administered at between about 10 mg to 4000 mg daily, optionally in divided doses), or

any combination thereof (e.g., optionally comprising any non-steroidal anti-inflammatory drug (NSAID), e.g., comprising aspirin, diclofenac; diflunisal, etodolac, fenoprofen, flurbiprofen, ibuprofen, indomethacin, ketoprofen; ketorolac, meclofenamate, mefenamic acid, meloxicam, nabumetone, naproxen, oxaprozin, piroxicam, salsalate, sulindac, tolmetin, a COX-2 inhibitor (e.g., a COX-2-selective inhibitor) or a combination thereof, wherein optionally the COX-2-selective inhibitor comprises celecoxib rofecoxib, etoricoxib, valdecoxib, parecoxib, meloxicam or lumiracoxib),

thereby ameliorating, diminishing, treating, blocking or preventing the systemic inflammation, or the inflammatory response, or the inflammatory response secondary to a disease, condition, contamination, poisoning, or infection, or a viral infection and/or a reactivation.

In alternative embodiments, the methods of the invention comprise ameliorating, diminishing, treating, blocking or preventing: an inflammation associated with a herpes virus infection, a human herpes virus-8 (HHV-8) infection, or a Kaposi's Sarcoma Herpes Virus infection; or an inflammation associated with a hyperproliferative skin disorder, Kaposi's Sarcoma, an inflammation secondary to HIV-induced immunosuppression, a B-cell disorder, Castleman's Disease, or Multicentric Castleman's Disease (MCD).

In alternative embodiments, the methods of the invention further comprise administering:

(a) a compound, a pharmaceutical composition or a formulation or therapy comprising:

an antiviral or an anti-retroviral agent or therapeutic,

a nucleoside reverse transcriptase inhibitor (optionally zidovudine, optionally administered at between about 100 mg to 4000 mg daily, optionally in divided doses,

a lamivudine, optionally administered at between about 100 mg to 600 mg daily, optionally in divided doses),

a non-nucleoside reverse transcriptase inhibitor (optionally nevirapine, optionally administered at between about 10 mg to 2000 mg daily, optionally in divided doses; or an efavirenz (optionally SUSTIVA™ or STOCRIN™), optionally administered at between about 10 mg to 2400 mg daily, optionally in divided doses),

a protease inhibitor,

an indinavir (optionally CRIXIVAN™), optionally administered at between about 100 mg to 4000 mg daily, optionally in divided doses,

a ritonavir (optionally NORVIR™) optionally administered at between about 100 mg to 2400 mg daily, optionally in divided doses),

an antiherpesvirus agent, or an antiherpesvirus agent capable of blocking viral replication and shedding of human herpes virus, wherein the herpesvirus is HHV-1 (Herpes Simplex Virus, or HSV 1), HHV-2 (Herpes Simplex Virus-2 or HVS2), HHV-3 (Varicella Zoster), HHV-4 (Epstein-Barr Virus, EBV), HHV-5 (cytomegalovirus, CMV), HHV-6 (roseolovirus, or herpes lymphotrophic virus), HHV-7 (roseolovirus), HHV-8 (Human Herpes Virus-8, also known as Kaposi's Sarcoma Herpes Virus, “KSHV”),

an ganciclovir (optionally CYTOVENE™; optionally administered at between about 1 mg to 4500 mg daily, optionally in divided doses) and its prodrug valganciclovir (optionally VALCYTE™; optionally administered at between about 100 mg to 4500 mg daily, optionally in divided doses),

an acyclovir (optionally ZOVIRAX™; optionally administered at between about 100 mg to 8000 mg daily, optionally in divided doses) and its prodrug valacyclovir (optionally VALTREX™, optionally administered at between about 1 g to 10 g per day, optionally in divided doses),

an cidofovir (optionally VISTIDE™; optionally administered at between about 1 mg/kg to 400 mg/kg daily, optionally by intravenous infusion),

a famciclovir (optionally FAMVIR™; optionally administered at between about 10 mg to 4000 mg daily, optionally in divided doses),

a penciclovir (optionally DENAVIR™; optionally administered at between about 10 mg to 400 mg daily, optionally in divided doses),

a foscarnet (optionally FOSCAVIR™; optionally administered at between about 10 mg/kg to 400 mg/kg daily, optionally by intravenous infusion),

an imiquimod (optionally ALDARA™; optionally administered at between about 10 mg to 400 mg daily, optionally in divided doses),

a ribavirin (optionally REBETOL™, optionally administered at between about 1 μg/kg/wk up to 10 μg/kg/wk, optionally given by subcutaneous injection),

an interferon-alpha (optionally ROFERON™; optionally administered at between about 1 MIU (about 20 ng) to 30 MIU, optionally weekly, optionally at approximately 600 ng weekly, optionally given in divided doses by subcutaneous injection, optionally comprising its pegolated derivatives, optionally PEG-INTRON™; optionally administered at between about 1 μg/kg up to 100 μg/kg weekly, optionally given by subcutaneous injection, or

any combination thereof;

(b) a compound, a pharmaceutical composition or a formulation or therapy comprising:

a compound, a pharmaceutical composition or a formulation or therapy targeting a cell infected with a virus or a human herpes virus-8 (HHV-8),

a rituximab (optionally RITUXAN™, or MABTHERA™) or other antibodies that target a CD20 antigen expressed on a B-cell, optionally administered at between about 10 mg/m² to 1000 mg/m² given by infusion, up to every two weeks),

an etoposide (optionally EPOSIN™, ETOPOPHOS™, VEPESID™, or VP-16™) or other inhibitors of a eukaryotic topoisomerase II (optionally based on podophyllotoxin) optionally administered at between about 10 mg/m² up to 100 mg/m², optionally taken orally in divided doses or given by intravenous infusion or any other route, or

any combination thereof;

(c) a compound, a pharmaceutical composition or a formulation or therapy comprising:

a compound, a pharmaceutical composition or a formulation or drug that modulates the immune system, or causes an HHV-8 reactivation due to an alteration in an immune system function,

a thalidomide, a lenalidomide, a pomalidomide, or a congener or analog (optionally THALOMID™, or REVLIMID™) optionally administered at between about 10 mg to 1000 mg daily, in divided doses, with or without concomitant glucocorticoid therapy,

a lenalidomide (optionally administered at between about 1 mg to 100 mg daily, with or without concomitant glucocorticoid therapy),

a pomalidomide (optionally administered at between about 0.1 mg to 40 mg daily, or every other day, with or without concomitant glucocorticoid therapy), or

any combination thereof;

(d) a compound, a pharmaceutical composition or a formulation or therapy comprising:

a compound, a pharmaceutical composition or a formulation or drug that modulates an immune system by interfering with a calcineurin/NFAT signaling in a T cells;

a cyclosporine, a cyclosporine A, or ciclosporin (optionally administered at between about 0.1 mg/kg/da to 20 mg/kg/da),

a tacrolimus (optionally FK-506™ or FUJIMYCIN™) optionally administered at between about 1 μg/kg/da up to 100 μg/kg/da by, optionally intravenous infusion,

a pimecrolimus (optionally ELIDEL™) optionally administered at between about 1 μg/kg/da up to 100 μg/kg/da, optionally by intravenous infusion, or

any combination thereof;

(e) a compound, a pharmaceutical composition or a formulation or therapy comprising:

a compound, a pharmaceutical composition or a formulation or drug that acts directly or indirectly as an anti-proliferative agent for a T cell,

a sirolimus or rapamycin (optionally RAPAMUNE™) optionally administered at between about 1 mg up to 100 mg/da, optionally by oral or intravenous route,

an inhibitor of a mammalian target of rapamycin (mTORs),

an azathioprine (optionally AZASAN™, IMURAN™, AZAMUN™, or IMUREL™) optionally administered at between about 0.1 mg/kg/da up to 10 mg/kg/da, optionally by oral, intravenous, or other route,

a mycophenolate mofetil or a mycophenolic acid (optionally CELLCEPT™ or MYFORTIC™) optionally administered at between about 100 mg up to 10 g/da, optionally by oral or intravenous, or

any combination thereof; or

(f) a compound, a pharmaceutical composition or a formulation or therapy comprising:

a compound, a pharmaceutical composition or a formulation or drug that interferes with signaling through an IL-6 cytokine pathway, or attenuates an immunomodulatory response induced by IL-6,

a monoclonal antibody that binds to an IL-6 receptor,

a tocilizumab (optionally administered at between about 1 mg/kg up to 20 mg/kg, optionally given by intravenous infusion, optionally as often as every 28 days; optionally a dosing schedule including daily infusions),

a monoclonal antibody that adsorbs an IL-6 peptide,

an elsilimomab (optionally B-E8™) administered at between about 1 mg/kg up to 10 mg/kg every 2 weeks), optionally given with or without concomitant glucocorticoid therapy, or

any combination thereof; or

(g) a compound, a pharmaceutical composition or a formulation or therapy comprising:

a compound, a pharmaceutical composition or a formulation or drug that is a cytotoxic drug,

a compound, a pharmaceutical composition or a formulation or drug that is a cancer chemotherapeutic,

a compound, a pharmaceutical composition or a formulation or drug that induces myelosuppression,

a compound, a pharmaceutical composition or a formulation or drug that is disrupts microtubule function,

a taxane (optionally paclitaxel or TAXOL™, or docetaxel or TAXOTERE™),

a polyether macrolide (optionally halichondrin B, or eribulin or HALAVEN™),

a compound, a pharmaceutical composition or a formulation or drug that inhibits a DNA methyltransferase activity,

an azacytidine, optionally a 5-azacytidine, optionally VIDAZA™, optionally administered at between about 10 mg/m²/da up to 300 mg/m²/da, optionally by subcutaneous injection, or optionally by intravenous infusion,

a decitabine, optionally a DACOGEN™, optionally administered at between about 1 mg/m² up to 100 mg/m² up to three times daily, optionally by intravenous infusion,

a depsipeptide drug that inhibits a histone deacetylase,

a romidepsin, optionally ISTODAX™, optionally administered at between about at 1 mg/m² up to 100 mg/m² weekly, optionally more or less frequently by intravenous infusion, or

any combination thereof.

In alternative embodiments, the invention provides products of manufacture comprising a pharmaceutical composition or a formulation, a blister package, a lidded blister or a blister card or packet, a clamshell, a tray or a shrink wrap, or a kit, comprising all ingredients to practice a method of the invention; and optionally further comprising instructions for use, wherein optionally the instructions comprise instructions for practicing all or part of a method of the invention.

In alternative embodiments, the invention provides uses of products of manufacture of the invention, or combinations thereof, in the manufacture of a medicament. In alternative embodiments, the invention provides uses of products of manufacture of the invention, or combinations thereof, in the manufacture of a medicament for ameliorating, diminishing, treating, blocking or preventing a systemic inflammation, or an inflammatory response, or an inflammatory response secondary to a disease, condition, contamination, poisoning, or infection, or a viral infection and/or a reactivation.

In alternative embodiments, the invention provides therapeutic combinations of drugs comprising or consisting of a combination of at least two compounds: wherein the at least two compounds comprise or consist of:

(1) (a) a therapeutic combination of drugs as set forth in the product of manufacture of the invention, or a combination thereof; or

(b) (i) a compound, pharmaceutical composition or formulation comprising an inhibitor of the sympathetic nervous system; and
(ii) a compound, pharmaceutical composition or formulation comprising an inhibitor of the lipid-derived autacoid system;

(2) the therapeutic combination of drugs of (1), wherein the inhibitor of the sympathetic nervous system comprises a beta adrenoceptor antagonist compound or drug; or

(3) the therapeutic combination of drugs of (1), wherein the inhibitor of the lipid-derived autacoid system comprises a non-selective or selective COX-2 inhibiting non-steroidal anti-inflammatory compound or drug.

In alternative embodiments, the invention provides combinations for ameliorating, diminishing, treating, blocking or preventing a systemic inflammation, or an inflammatory response, or an inflammatory response secondary to a disease, condition, contamination, poisoning, or infection, or a viral infection and/or a reactivation, comprising:

(1) (a) a therapeutic combination of drugs as set forth in the product of manufacture of the invention, or a combination thereof; or

(b) (i) a compound, pharmaceutical composition or formulation comprising an inhibitor of the sympathetic nervous system; and
(ii) a compound, pharmaceutical composition or formulation comprising an inhibitor of the lipid-derived autacoid system;

(2) the therapeutic combination of drugs of (1), wherein the inhibitor of the sympathetic nervous system comprises a beta adrenoceptor antagonist compound or drug; or

(3) the therapeutic combination of drugs of (1), wherein the inhibitor of the lipid-derived autacoid system comprises a non-selective or selective COX-2 inhibiting non-steroidal anti-inflammatory compound or drug.

In alternative embodiments, the invention provides compositions, pharmaceutical compositions or formulations, or any combination thereof, for use in ameliorating, diminishing, treating, blocking or preventing a systemic inflammation, or an inflammatory response, or an inflammatory response secondary to a disease, condition, contamination, poisoning, or infection, or a viral infection and/or a reactivation, comprising:

(1) (a) a therapeutic combination of drugs as set forth in the product of manufacture of the invention, or a combination thereof; or

(b) (i) a compound, pharmaceutical composition or formulation comprising an inhibitor of the sympathetic nervous system; and
(ii) a compound, pharmaceutical composition or formulation comprising an inhibitor of the lipid-derived autacoid system;

(2) the therapeutic combination of drugs of (1), wherein the inhibitor of the sympathetic nervous system comprises a beta adrenoceptor antagonist compound or drug; or

(3) the therapeutic combination of drugs of (1), wherein the inhibitor of the lipid-derived antacoid system comprises a non-selective or selective COX-2 inhibiting non-steroidal anti-inflammatory compound or drug.

The details of one or more aspects of the invention are set forth in the description below. Other features, objects, and advantages of the invention will be apparent from the description and from the claims.

All publications, patents and patent applications cited herein are hereby expressly incorporated by reference for all purposes.

### DETAILED DESCRIPTION

In alternative embodiments, the invention provides compositions, e.g., pharmaceutical compositions and preparations, formulations, kits and other products of manufacture, comprising: an inhibitor of the sympathetic nervous system; and an inhibitor of the lipid-derived autacoid system.

In alternative embodiments, exemplary drug combinations of the invention (at least an inhibitor of the sympathetic nervous system and an inhibitor of the lipid-derived autacoid system) are packaged together or separately in blister packs, lidded blisters or blister cards, or wrapped in paper, plastic or cellophane wrappers (e.g., a shrink wrap). In alternative embodiments, exemplary drug combinations of the invention comprise a combination regimen of the at least two active ingredients (i.e., an inhibitor of the sympathetic nervous system and an inhibitor of the lipid-derived autacoid system) designed to diminish systemic inflammation by targeting (inhibiting) two different, but convergent, signaling pathways, i.e., the sympathetic nervous system and the lipid-derived autacoid system.

In alternative embodiments, the compositions and methods of the invention (e.g., the combination of drugs (at least an inhibitor of the sympathetic nervous system and an inhibitor of the lipid-derived autacoid system) are used to ameliorate, diminish, treat, block or prevent an inflammatory response, e.g., an inflammatory response secondary to a disease, condition, contamination, poisoning, or infection, e.g., a viral infection and/or a reactivation.

In alternative embodiments, the compositions and methods of the invention (e.g., the combination of drugs, i.e., at least an inhibitor of the sympathetic nervous system and an inhibitor of the lipid-derived autacoid system) are used to ameliorate, diminish, treat, block or prevent the systemic inflammation that appears as a co-morbidity factor in certain viral infection, e.g., a human herpes virus-8 (HHV-8), or Kaposi's Sarcoma Herpes Virus. In alternative embodiments, the compositions and methods of the invention are used to ameliorate, diminish, treat, block or prevent the systemic inflammation associated with: a hyperproliferative skin disorder, or Kaposi's Sarcoma (KS), or a KS observed secondary to HIV-induced immunosuppression, a B-cell disorder, Castleman's Disease (CD), and/or a form of CD that is more refractory to treatment known as Multicentric Castleman's Disease (MCD).

In alternative embodiments, compositions, including pharmaceutical compositions and preparations, formulations, kits and other products of manufacture, e.g., exemplary drug and formulation combinations are packaged together or separately in products of manufacture, e.g., as blister packs or packettes, lidded blisters or blister cards, or wrapped in paper, aluminum, plastic or cellophane wrappers (e.g., a shrink wrap), comprising combinations of beneficial ingredients of the invention.

### Methods of Administration

This invention provides compositions (e.g., for use in the exemplary combinations of drug of the invention), e.g., pharmaceutical compositions, preparations and kits, that can be administered by several routes, including intravenous, topical and oral, or combinations thereof.

For example, one embodiment comprises a product of manufacture comprising a pharmaceutical composition or a formulation, a blister package, a lidded blister or a blister card or packet, a clamshell, a tray or a shrink wrap, or a kit, comprising at least the combination of an inhibitor of the sympathetic nervous system and an inhibitor of the lipid-derived autacoid system. Each ingredient can be either separately packaged, or can be formulated as one unit dose, e.g., as one tube (e.g., with gel, lotion etc.), ampoule, blister packette and the like.

In alternative embodiments, this invention provides forms of compositions, preparations and kits that can be administered by inhalation, infusion or injection, (e.g., intraperitoneal, intramuscular, subcutaneous, intra-aural, intra-articular, intra-mammary, etc.), topical application (e.g., on areas, such as eyes, cars, skin or on afflictions such as wounds, burns, etc.), and by absorption through epithelial or mucocutaneous linings (e.g. vaginal and other epithelial linings, gastrointestinal mucosa, etc.). Methods are known for making compositions, preparations and kits containing the present components that are suitable for each of these methods of administration as well as other methods of administration that are known in the art.

In alternative embodiments, this invention provides compositions, preparations and kits in liquid forms that can be administered orally. The compositions, preparations and kits can be also prepared as capsules, gels, geltabs, tablets, powders, sprays, aerosols, pellets (e.g. for animal consumption), suppositories, lotions, patches or adhesives (e.g., for the skin), or creams and ointments. The compositions, preparations and kits can be also prepared as physiological solutions suitable for I.V. administration or other parenteral administration.

In one aspect, a multi-ingredient kit of the invention comprises (contains) two or more ingredients in approximately equal amounts. An amount may be determined, e.g. by mass or by weight or by molar amount. In another aspect, a multi-ingredient kit may contain two or more ingredients in unequal amounts. In another aspect, a multi-ingredient kit may contain two or more ingredients in approximately equal amounts as well as one or more ingredients that are not in unequal amounts.

Thus, in alternative embodiments, a multi-ingredient kit may contain two or more ingredients in approximately equimolar amounts. In another embodiment, a multi-ingredient kit may contain two or more ingredients that are not in equimolar amounts. In another aspect, a multi-ingredient kit may contain two or more ingredients that are in approximately equimolar amounts as well as one or more ingredients that are not in equimolar amounts.

In another embodiment, said multi-ingredient kit may contain two or more ingredients that are admixed. In another aspect, said multi-ingredient kit may contain two or more ingredients that are not admixed. In another aspect, said multi-ingredient kit may contain two or more ingredients that are partially admixed. In another aspect, said multi-ingredient kit may contain two or more ingredients that are at least partially admixed, as well as one or more ingredients that are not admixed. An ingredient in a multi-ingredient kit may be liquid forms that can be administered orally.

In another embodiment, an ingredient in a multi-ingredient kit may also be in delivery forms such as capsules, tablets, powders, sprays, aerosols, pellets (e.g. for animal consumption), suppositories, or creams and ointments. An ingredient in a multi-ingredient kit may also be in delivery forms such as physiological solutions suitable for I.V. administration or other parenteral administration.

In another embodiment, the ingredients in a multi-ingredient kit may be separated by physically compartmentalization (e.g. in separate compartments that are part of said kit, where said kit is a multi-compartment kit). Thus, for example, it is provided that the ingredients may be admixed or not admixed. For example, a single pill or capsule may contain more than one key ingredient (e.g. (at least the combination of an inhibitor of the sympathetic nervous system and an inhibitor of the lipid-derived autacoid system). Alternatively, separate compartments, as may be found in a “blister pack” type of packaging, may contain different ingredients.

### Packaging

The invention provides compositions, including preparations, formulations and/or kits, comprising combinations of ingredients, as described herein, e.g., at least the combination of an inhibitor of the sympathetic nervous system and an inhibitor of the lipid-derived autacoid system. In one aspect, each member of the combination of ingredients is manufactured in a separate package, kit or container, or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills. etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one aspect, a blister pack of the invention also comprises a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two components (e.g., is a multi-ingredient combination of drugs of the invention): a thermoformed “blister” which houses the product (e.g., a pharmaceutical combination of the invention), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

### Blister Packaging

In alternative embodiments, combinations of the invention (at least the combination of an inhibitor of the sympathetic nervous system and an inhibitor of the lipid-derived autacoid system) can comprise the packaging of the therapeutic drug combinations of the invention, alone or in combination, as “blister packages” or as a plurality of packettes, including as lidded blister packages, lidded blister or blister card or packets or packettes, or a shrink wrap.

In alternative embodiments, laminated aluminum foil blister packs are used, e.g., for the preparation of drugs designed to dissolve immediately in the mouth of a patient. This exemplary process comprises having the drug combinations of the invention prepared as an aqueous solution(s) which are dispensed (e.g., by measured dose) into an aluminum (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminum (e.g., alufoil) lidding material. In one aspect, hermetically-sealed high barrier aluminum (e.g., alufoil) laminates are used. In one aspect, any of the invention's products of manufacture, including kits or blister packs, use foil laminations and strip packs, stick packs, sachets and pouches, peelable and non-peelable laminations combining foil, paper, and film for high barrier packaging.

In alternative embodiments, any of the invention's products of manufacture, including kits or blister packs, include memory aids to help remind patients when and how to take the drug. This safeguards the drug's efficacy by protecting each pill until it's taken; gives the product or kit portability, makes it easy to take a dose anytime or anywhere.

In alternative embodiments, the invention provides compliance kits; which can be used to solve several problems that make patient adherence of complex regimens problematic. In alternative embodiments, compliance kits of the invention are designed to solve the various patient adherence problems including: (1) eliminate difficulty in assembling components: the patient does not have to go to store, figure out which products to buy and then products home, e.g., every month (2) eliminate self-pay/co-pay for the components that discourage usage (3) encourage adherence with instructional video/packaging that explain why good idea (4) encourage adherence with phone calls from specialty pharmacy to answer question and review usage (5) encourage adherence with smart packaging/embedded chips to track usage and alert specialty pharmacy/care givers/physician to non-adherence so corrective measure can be taken.

A number of aspects of the invention have been described. Nevertheless, it will be understood that various modifications may be made without departing from the spirit and scope of the invention. Accordingly, other aspects are within the scope of the following claims.

What is claimed is:

1. A product of manufacture comprising a compound, a pharmaceutical composition or a formulation, a blister package, a lidded blister or a blister card or packet, a clamshell, a tray or a shrink wrap, or a kit, comprising:
(a) a compound, pharmaceutical composition or formulation comprising an inhibitor of the sympathetic nervous system; and
(b) a compound, pharmaceutical composition or formulation comprising an inhibitor of the lipid-derived autacoid system.

2. The product of manufacture of claim 1, further comprising a compound, a pharmaceutical composition or a formulation or therapy comprising:
an anti-viral or an anti-retroviral agent or therapeutic,
a nucleoside reverse transcriptase inhibitor (optionally zidovudine, optionally administered at between about 100 mg to 4000 mg daily, optionally in divided doses,
a lamivudine, optionally administered at between about 100 mg to 600 mg daily, optionally in divided doses),
a non-nucleoside reverse transcriptase inhibitor (optionally nevirapine, optionally administered at between about 10 mg to 2000 mg daily, optionally in divided doses; or an efavirenz, optionally administered at between about 10 mg to 2400 mg daily, optionally in divided doses),
a protease inhibitor,
an indinavir (optionally CRIXIVAN™), optionally administered at between about 100 mg to 4000 mg daily, optionally in divided doses,
a ritonavir (optionally NORVIR™) optionally administered at between about 100 mg to 2400 mg daily, optionally in divided doses,
an antiherpesvirus agent, or an antiherpesvirus agent capable of blocking viral replication and shedding of human herpes virus, wherein the herpesvirus is HHV-1 (Herpes Simplex Virus, or HSV 1), HHV-2 (Herpes Simplex Virus-2 or HVS2), HHV-3 (Varicella Zoster), HHV-4 (Epstein-Barr Virus, EBV), HHV-5 (cytomegalovirus, CMV), HHV-6 (roseolovirus, or herpes lymphotrophic virus), HHV-7 (roseolovirus), HHV-8 (Human Herpes Virus-8, also known as Kaposi's Sarcoma Herpes Virus, “KSHV”),
an ganciclovir (optionally CYTOVENE™; optionally administered at between about 1 mg to 4500 mg daily, optionally in divided doses) and its prodrug valganciclovir (optionally VALCYTE™; optionally administered at between about 100 mg to 4500 mg daily, optionally in divided doses),
an acyclovir (optionally ZOVIRAX™; optionally administered at between about 100 mg to 8000 mg daily, in divided doses) and its prodrug valacyclovir (optionally VALTREX™, optionally administered at between about 1 g to 10 g per day, optionally in divided doses),
an cidofovir (optionally VISTIDE™; optionally administered at between about 1 mg/kg to 400 mg/kg daily, optionally by intravenous infusion),
a famciclovir (optionally FAMVIR™; optionally administered at between about 10 mg to 4000 mg daily, optionally in divided doses),
a penciclovir (optionally DENAVIR™; optionally administered at between about 10 mg to 400 mg daily, optionally in divided doses),
a foscarnet (optionally FOSCAVIR™; optionally administered at between about 10 mg/kg to 400 mg/kg daily, optionally by intravenous infusion),
an imiquimod (optionally ALDARA™; optionally administered at between about 10 mg to 400 mg daily, optionally in divided doses),
a ribavirin (optionally REBETOL™, optionally administered at between about 1 μg/kg/wk up to 10 μg/kg/wk, optionally given by subcutaneous injection),
an interferon-alpha (optionally ROFERON™; optionally administered at between about 1 MIU (about 20 ng) to 30 MIU, optionally weekly, optionally at approximately 600 ng weekly, optionally given in divided doses by subcutaneous injection, optionally comprising its pegolated derivatives, optionally PEG-INTRON™; optionally administered at between about 1 μg/kg up to 100 μg/kg weekly, optionally given by subcutaneous injection, or
any combination thereof.

3. A method for ameliorating, diminishing, treating, blocking or preventing a systemic inflammation, or an inflammatory response, or an inflammatory response secondary to a disease, condition, contamination, poisoning, or infection, or a viral infection and/or a reactivation, comprising:
(a) administering to an individual, a patient or an animal in need thereof a product of manufacture, a pharmaceutical composition or a formulation, a blister package, a lidded blister or a blister card or packet, a clamshell, a tray or a shrink wrap, or a kit, of claim 1;
(b) administering to an individual, a patient or an animal in need thereof:
(i) a compound, pharmaceutical composition or formulation comprising an inhibitor of the sympathetic nervous system; and
(ii) a compound, pharmaceutical composition or formulation comprising an inhibitor of the lipid-derived autacoid system;
(c) the method of (b), wherein the inhibitor of the sympathetic nervous system comprises a beta adrenoceptor antagonist compound or drug;
(d) the method of (b), wherein the inhibitor of the lipid-derived autacoid system comprises a non-selective or selective COX-2 inhibiting non-steroidal anti-inflammatory compound or drug; or
(e) the method of (c), wherein the beta adrenoceptor antagonist comprises:
a propranolol, or a 2-Propanol, 1-[(1-methylethyl)amino]-3-(1-naphthalenyloxy)-, hydrochloride, or equivalent (optionally INDERAL™, AVLOCARDYL™, AVLOCARDYL RETARD™, DERALIN™, DOCITON™, INDERALICI™, INNOPRAN XL™, SUMIAL™, or ANAPRILINUM™) optionally administered at between about 10 mg up to 800 mg daily, optionally in divided doses,
a nadolol (optionally CORGARD™, ANABET™, SOLGOL™, CORZIDE™, ALTI-NADOLOL™, APO-NADOL™, or NOVO-NADOLOL™) optionally administered at between about 1 mg up to 400 mg once daily,
a timolol or timolol maleate (optionally administered at between about 1 mg up to 400 mg daily, optionally in divided doses),
a pindolol (optionally VISKEN™, BETAPINDOL™, BLOCKIN L™, BLOCKLIN L™, CALVISKEN™, CARDILATE™, DECRETEN™, DURAPINDOL™, GLAUCO-VISKEN™, PECTOBLOC™, PINBETOL™, PRINDOLOL™, or PYNASTIN™) optionally administered at between about 1 mg up to 200 mg daily, optionally in divided doses),
a labetalol (optionally NORMODYNE™, TRANDATE™, or NORMOZYDE™) optionally administered at between about 10 mg up to 4000 mg daily, optionally in divided doses),
a beta-1-selective drug,
a metoprolol (optionally administered at between about 10 mg up to 800 mg daily, optionally in divided doses),
an atenolol (optionally TENORMIN™) optionally administered at between about 1 mg up to 200 mg daily),
an acebutolol (optionally SECTRAL™, or PRENT™) optionally administered at between about 10 mg up to 2400 mg daily, optionally in divided doses),
an alpha-beta non-selective agent,
a carvedilol (optionally COREG™, DILATREND™, EUCARDIC™, CARLOC™, CIPLA™, or COREG CR™) optionally administered at between about 1 mg up to 400 mg daily, optionally in divided doses), or
any combination thereof (e.g., comprising at least one atenolol, nadolol, metoprolol, propranolol, carteolol, carvedolol, labetalol, oxprenolol, penbutolol, pindolol, sotalol, timolol or a combination thereof); or
(f) the method of (d), wherein the non-selective or selective COX-2 inhibiting non-steroidal anti-inflammatory drug comprises:
a diaryl furanone (optionally a rofecoxib, optionally VIOXX™, CEOXX™, or CEEOXX™, optionally administered at between about 1 mg to 100 mg daily),
a diaryl pyrazole (optionally a celecoxib, optionally COBIX™, CELCOXX™, or SELECAP™) optionally administered at between about 1 mg to 800 mg daily),
an indole acetate (optionally a etodolac, optionally LODINE SR™, or ECCOXOLAC™, optionally administered at between about 10 mg to 2000 mg daily, optionally in divided doses),
a sulfonamide (optionally a nimensulide, optionally AULIN™, MESULIDE™, or NIMED™) optionally administered at between about 10 mg to 1000 mg daily),
a salicylate (optionally a acetyl salicylic acid or aspirin, optionally administered at between about 40 mg to 4000 mg daily, optionally in divided doses),
an indole or an indene acetic acid (optionally an indomethacin, optionally INDOCIN™, INDOCID™, INDOCHRON E-R™, or INDOCIN-S™, optionally administered at between about 10 mg to 400 mg daily, optionally in divided doses),
a heteroaryl acetic acid (optionally a diclofenac, optionally CATAFLAM™, or ZIPSOR™), optionally administered at between about 10 mg to 400 mg daily, optionally in divided doses),
an arylpropionic acid, optionally an ibuprofen (optionally BRUFEN™, NUROFEN™, ADVIL™, or NUPRIN™), optionally administered at between about 10 mg to 4000 mg daily, optionally in divided doses;
a naproxen, optionally ALEVE™, ANAPROX™, ANTALGIN™, FEMINAX ULTRA™, FLANAX™, INZA™, MIDOL EXTENDED RELIEF™, MIRANAXV, NALGESIN™, NAPOSIN™, NAPRELAN™, NAPROGESIC™, NAPROSYN™, NAROCIN™, PROXEN™, SYNFLEX™, or XENOBID™, optionally at 10 mg to 4000 mg daily, optionally in divided doses,
a fenamate, optionally a mefanamic acid, optionally PONSTEL™, optionally administered at between about 100 mg to 4000 mg daily, optionally in divided doses,
an enolate (optionally a meloxicam, optionally MOVALIS™, MELOX™, RECOXA™, MOBIC™, MOBEC™, MOBICOX™, TENARON™, ILACOX™, MAVICAM™, MELOCAM™, or ARTRIFLAM™) optionally administered at between about 1 mg to 100 mg daily; optionally administered at between about piroxicam at 1 mg to 100 mg daily),
an alkanone (optionally a nabumetone, optionally RELAFEN™, RELIFEX™, or GAMBARAN™) optionally administered at between about 10 mg to 4000 mg daily, optionally in divided doses), or
any combination thereof (e.g., optionally comprising any non-steroidal anti-inflammatory drug (NSAID), e.g., comprising aspirin, diclofenac; diflunisal, etodolac, fenoprofen, flurbiprofen, ibuprofen, indomethacin, ketoprofen; ketorolac, meclofenamate, mefenamic acid, meloxicam, nabumetone, naproxen, oxaprozin, piroxicam, salsalate, sulindac, tolmetin, a COX-2 inhibitor (e.g., a COX-2-selective inhibitor) or a combination thereof, wherein optionally the COX-2-selective inhibitor comprises celecoxib rofecoxib, etoricoxib, valdecoxib, parecoxib, meloxicam or lumiracoxib),
thereby ameliorating, diminishing, treating, blocking or preventing the systemic inflammation, or the inflammatory response, or the inflammatory response secondary to a disease, condition, contamination, poisoning, or infection, or a viral infection and/or a reactivation.

4. The method claim 3, wherein the method comprises ameliorating, diminishing, treating, blocking or preventing: an inflammation associated with a herpes virus infection, a human herpes virus-8 (HHV-8) infection, or a Kaposi's Sarcoma Herpes Virus infection; or an inflammation associated with a hyperproliferative skin disorder, Kaposi's Sarcoma, an inflammation secondary to HIV-induced immunosuppression, a B-cell disorder, Castleman's Disease, or Multicentric Castleman's Disease (MCD).

5. The method of claim 3, further comprising administering
(a) a compound, a pharmaceutical composition or a formulation or therapy comprising:
an antiviral or an anti-retroviral agent or therapeutic,
a nucleoside reverse transcriptase inhibitor (optionally zidovudine, optionally administered at between about 100 mg to 4000 mg daily, optionally in divided doses,
a lamivudine, optionally administered at between about 100 mg to 600 mg daily, optionally in divided doses),
a non-nucleoside reverse transcriptase inhibitor (optionally nevirapine, optionally administered at between about 10 mg to 2000 mg daily, optionally in divided doses; or an efavirenz (optionally SUSTIVA™ or STOCRIN™), optionally administered at between about 10 mg to 2400 mg daily, optionally in divided doses),
a protease inhibitor,
an indinavir (optionally CRIXIVAN™), optionally administered at between about 100 mg to 4000 my daily, optionally in divided doses,
a ritonavir (optionally NORVIR™) optionally administered at between about 100 mg to 2400 mg daily, optionally in divided doses),
an antiherpesvirus agent, or an antiherpesvirus agent capable of blocking viral replication and shedding of human herpes virus, wherein the herpesvirus is HHV-1 (Herpes Simplex Virus, or HSV 1), HHV-2 (Herpes Simplex Virus-2 or HVS2), HHV-3 (Varicella Zoster), HHV-4 (Epstein-Barr Virus, EBV), HHV-5 (cytomegalovirus, CMV), HHV-6 (roseolovirus, or herpes lymphotrophic virus), HHV-7 (roseolovirus), HHV-8 (Human Herpes Virus-8, also known as Kaposi's Sarcoma Herpes Virus, “KSHV”),
an ganciclovir (optionally CYTOVENE™; optionally administered at between about 1 mg to 4500 mg daily, optionally in divided doses) and its prodrug valganciclovir (optionally VALCYTE™; optionally administered at between about 100 mg to 4500 mg daily, optionally in divided doses),
an acyclovir (optionally ZOVIRAX™; optionally administered at between about 100 mg to 8000 mg daily, optionally in divided doses) and its prodrug valacyclovir (optionally VALTREX™, optionally administered at between about 1 g to 10 g per day, optionally in divided doses),
an cidofovir (optionally VISTIDE™; optionally administered at between about 1 mg/kg to 400 mg/kg daily, optionally by intravenous infusion),
a famciclovir (optionally FAMVIR™; optionally administered at between about 10 mg to 4000 mg daily, optionally in divided doses),
a penciclovir (optionally DENAVIR™; optionally administered at between about 10 mg to 400 mg daily, optionally in divided doses),
a foscarnet (optionally FOSCAVIR™; optionally administered at between about 10 mg/kg to 400 mg/kg daily, optionally by intravenous infusion),
an imiquimod (optionally ALDARA™; optionally administered at between about 10 mg to 400 mg daily, optionally in divided doses),
a ribavirin (optionally REBETOL™, optionally administered at between about 1 μg/kg/wk up to 10 μg/kg/wk, optionally given by subcutaneous injection),
an interferon-alpha (optionally ROFERON™; optionally administered at between about 1 MIU (about 20 ng) to 30 MIU, optionally weekly, optionally at approximately 600 ng weekly, optionally given in divided doses by subcutaneous injection, optionally comprising its pegolated derivatives, optionally PEG-INTRON™; optionally administered at between about 1 μg/kg up to 100 μg/kg weekly, optionally given by subcutaneous injection, or
any combination thereof;
(b) a compound, a pharmaceutical composition or a formulation or therapy comprising:
a compound, a pharmaceutical composition or a formulation or therapy targeting a cell infected with a virus or a human herpes virus-8 (HHV-8),
a rituximab (optionally RITUXAN™, or MABTHERA™) or other antibodies that target a CD20 antigen expressed on a B-cell, optionally administered at between about 10 mg/m² to 1000 mg/m² given by infusion, up to every two weeks),
an etoposide (optionally EPOSIN™, ETOPOPHOS™, VEPESID™, or VP-16™) or other inhibitors of a eukaryotic topoisomerase II (optionally based on podophyllotoxin), optionally administered at between about 10 mg/m² up to 100 mg/m², optionally taken orally in divided doses or given by intravenous infusion or any other route, or
any combination thereof;
(c) a compound, a pharmaceutical composition or a formulation or therapy comprising:
a compound, a pharmaceutical composition or a formulation or drug that modulates the immune system, or causes an HHV-8 reactivation due to an alteration in an immune system function,
a thalidomide, a lenalidomide, a pomalidomide, or a congener or analog (optionally THALOMID™, or REVLIMID™) optionally administered at between about 10 mg to 1000 mg daily, in divided doses, with or without concomitant glucocorticoid therapy,
a lenalidomide (optionally administered at between about 1 mg to 100 mg daily, with or without concomitant glucocorticoid therapy),
a pomalidomide (optionally administered at between about 0.1 mg to 40 mg daily, or every other day, with or without concomitant glucocorticoid therapy), or
any combination thereof;
(d) a compound, a pharmaceutical composition or a formulation or therapy comprising:
a compound, a pharmaceutical composition or a formulation or drug that modulates an immune system by interfering with a calcineurin, NFAT signaling in a T cells;
a cyclosporine, a cyclosporine A, or ciclosporin (optionally administered at between about 0.1 mg/kg/da to 20 mg/kg/da),
a tacrolimus (optionally FK-506™ or FUJIMYCIN™) optionally administered at between about 1 μg/kg/da up to 100 μg/kg/da by, optionally intravenous infusion,
a pimecrolimus (optionally ELIDEL™) optionally administered at between about 1 μg/kg/da up to 100 μg/kg/da, optionally by intravenous infusion, or
any combination thereof;
(e) a compound, a pharmaceutical composition or a formulation or therapy comprising:
a compound, a pharmaceutical composition or a formulation or drug that acts directly or indirectly as an anti-proliferative agent for a T cell,
a sirolimus or rapamycin (optionally RAPAMUNE™) optionally administered at between about 1 mg up to 100 mg/da, optionally by oral or intravenous route,
an inhibitor of a mammalian target of rapamycin (mTORs),
an azathioprine (optionally AZASAN™, IMURAN™, AZAMUN™, or IMUREL™) optionally administered at between about 0.1 mg/kg/da up to 10 mg/kg/da, optionally by oral, intravenous, or other route,
a mycophenolate mofetil or a mycophenolic acid (optionally CELLCEPT™ or MYFORTIC™) optionally administered at between about 100 mg up to 10 g/da, optionally by oral or intravenous, or
any combination thereof; or
(f) a compound, a pharmaceutical composition or a formulation or therapy comprising:
a compound, a pharmaceutical composition or a formulation or drug that interferes with signaling through an IL-6 cytokine pathway, or attenuates an immunomodulatory response induced by IL-6,
a monoclonal antibody that binds to an IL-6 receptor,
a tocilizumab (optionally administered at between about 1 mg/kg up to 20 mg/kg, optionally given by intravenous infusion, optionally as often as every 28 days; optionally a dosing schedule including daily infusions),
a monoclonal antibody that adsorbs an IL-6 peptide,
an elsilimomab (optionally B-E8™) administered at between about 1 mg/kg up to 10 mg/kg every 2 weeks), optionally given with or without concomitant glucocorticoid therapy, or
any combination thereof; or
(g) a compound, a pharmaceutical composition or a formulation or therapy comprising:
a compound, a pharmaceutical composition or a formulation or drug that is a cytotoxic drug,
a compound, a pharmaceutical composition or a formulation or drug that is a cancer chemotherapeutic,
a compound, a pharmaceutical composition or a formulation or drug that induces myelosuppression,
a compound, a pharmaceutical composition or a formulation or drug that is disrupts microtubule function,
a taxane (optionally paclitaxel or TAXOL™, or docetaxel or TAXOTERE™),
a polyether macrolide (optionally halichondrin B, or eribulin or HALAVEN™),
a compound, a pharmaceutical composition or a formulation or drug that inhibits a DNA methyltransferase activity,
an azacytidine, optionally a 5-azacytidine, optionally VIDAZA™, optionally administered at between about 10 mg/m²/da up to 300 mg/m²/da, optionally by subcutaneous injection, or optionally by intravenous infusion,
a decitabine, optionally a DACOGEN™, optionally administered at between about 1 mg/m² up to 100 mg/m² up to three times daily, optionally by intravenous infusion,
a depsipeptide drug that inhibits a histone deacetylase,
a romidepsin, optionally ISTODAX™, optionally administered at between about at 1 mg/m² up to 100 mg/m² weekly, optionally more or less frequently by intravenous infusion, or
any combination thereof.

6. A product of manufacture comprising a pharmaceutical composition or a formulation, a blister package, a lidded blister or a blister card or packet, a clamshell, a tray or a shrink wrap, or a kit, comprising all ingredients to practice the method of claim 3.

7. The product of manufacture of claim 6, further comprising instructions for use, wherein the instructions comprise instructions for practicing all or part of the method of claim 3.

8. A therapeutic combination of drugs comprising or consisting of a combination of at least two compounds: wherein the at least two compounds comprise or consist of
(1) (a) a therapeutic combination of drugs as set forth in the product of manufacture of claim 1; or
(b) (i) a compound, pharmaceutical composition or formulation comprising an inhibitor of the sympathetic nervous system; and
(ii) a compound, pharmaceutical composition or formulation comprising an inhibitor of the lipid-derived autacoid system;
(2) the therapeutic combination of drugs of (1), wherein the inhibitor of the sympathetic nervous system comprises a beta adrenoceptor antagonist compound or drug; or
(3) the therapeutic combination of drugs of (1), wherein the inhibitor of the lipid-derived antacoid system comprises a non-selective or selective COX-2 inhibiting non-steroidal anti-inflammatory compound or drug.

9. A combination for ameliorating, diminishing, treating, blocking or preventing a systemic inflammation, or an inflammatory response, or an inflammatory response secondary to a disease, condition, contamination, poisoning, or infection, or a viral infection and/or a reactivation, comprising:
(1) (a) a therapeutic combination of drugs as set forth in the product of manufacture of claim 1; or
(b) (i) a compound, pharmaceutical composition or formulation comprising an inhibitor of the sympathetic nervous system; and
(ii) a compound, pharmaceutical composition or formulation comprising an inhibitor of the lipid-derived autacoid system;
(2) the therapeutic combination of drugs of (1), wherein the inhibitor of the sympathetic nervous system comprises a beta adrenoceptor antagonist compound or drug; or
(3) the therapeutic combination of drugs of (1), wherein the inhibitor of the lipid-derived autacoid system comprises a non-selective or selective COX-2 inhibiting non-steroidal anti-inflammatory compound or drug.

10. A composition, a pharmaceutical composition or formulation, or a combination, for use in ameliorating, diminishing, treating, blocking or preventing a systemic inflammation, or an inflammatory response, or an inflammatory response secondary to a disease, condition, contamination, poisoning, or infection, or a viral infection and/or a reactivation, comprising:
(1) (a) a therapeutic combination of drugs as set forth in the product of manufacture of claim 1; or
(b) (i) a compound, pharmaceutical composition or formulation comprising an inhibitor of the sympathetic nervous system; and
(ii) a compound, pharmaceutical composition or formulation comprising an inhibitor of the lipid-derived autacoid system;
(2) the therapeutic combination of drugs of (1), wherein the inhibitor of the sympathetic nervous system comprises a beta adrenoceptor antagonist compound or drug; or
(3) the therapeutic combination of drugs of (1), wherein the inhibitor of the lipid-derived autacoid system comprises a non-selective or selective COX-2 inhibiting non-steroidal anti-inflammatory compound or drug.

11. The product of manufacture of claim 1, wherein the inhibitor of the sympathetic nervous system comprises a beta adrenoceptor antagonist compound or drug.

12. The product of manufacture of claim 1, wherein the inhibitor of the lipid-derived autacoid system comprises a non-selective or selective COX-2 inhibiting non-steroidal anti-inflammatory compound or drug.

13. The product of manufacture of claim 11, wherein the beta adrenoceptor antagonist comprises:
a propranolol, or a 2-Propanol, 1-[(1-methylethyl)amino]-3-(1-naphthalenyloxy)-, hydrochloride, or equivalent (optionally INDERAL™, AVLOCARDYL™, AVLOCARDYL RETARD™, DERALIN™, DOCITON™, INDERALICI™, INNOPRAN XL™, SUMIAL™, or ANAPRILINUM™) optionally administered at between about 10 mg up to 800 mg daily, optionally in divided doses,
a nadolol (optionally CORGARD™, ANABET™, SOLGOL™, CORZIDE™, ALTI-NADOLOL™, APO-NADOL™, or NOVO-NADOLOL™) optionally administered at between about 1 mg up to 400 mg once daily,
a timolol or timolol maleate (optionally administered at between about 1 mg up to 400 mg daily, optionally in divided doses),
a pindolol (optionally VISKEN™, BETAPINDOL™, BLOCKIN L™, BLOCKLIN L™, CALVISKEN™, CARDILATE™, DECRETEN™, DURAPINDOL™, GLAUCO-VISKEN™, PECTOBLOC™, PINBETOL™, PRINDOLOL™, or PYNASTIN™) optionally administered at between about 1 mg up to 200 mg daily, optionally in divided doses),
a labetalol (optionally NORMODYNE™, TRANDATE™, or NORMOZYDE™) optionally administered at between about 10 mg up to 4000 mg daily, optionally in divided doses),
a beta-1-selective drug,
a metoprolol (optionally administered at between about 10 mg up to 800 mg daily, optionally in divided doses),
an atenolol (optionally TENORMIN™) optionally administered at between about 1 mg up to 200 mg daily),
an acebutolol (optionally SECTRAL™, or PRENT™) optionally administered at between about 10 mg up to 2400 mg daily, optionally in divided doses),
an alpha-beta non-selective agent,
a carvedilol (optionally COREG™, DILATREND™, EUCARDIC™, CARLOC™, CIPLA™, or COREG CR™) optionally administered at between about 1 mg up to 400 mg daily, optionally in divided doses), or
any combination thereof (e.g., comprising at least one atenolol, nadolol, metoprolol, propranolol, carteolol, carvedolol, labetalol, oxprenolol, penbutolol, pindolol, sotalol, timolol or a combination thereof).

14. The product of manufacture of claim 12, wherein the non-selective or selective COX-2 inhibiting non-steroidal anti-inflammatory drug comprises:
a diaryl furanone (optionally a rofecoxib, optionally VIOXX™, CEOXX™, or CEEOXX™, optionally administered at between about 1 mg to 100 mg daily),
a diaryl pyrazole (optionally a celecoxib, optionally COBIX™, CELCOXX™, or SELECAP™) optionally administered at between about 1 mg to 800 mg daily),
an indole acetate (optionally a etodolac, optionally LODINE SR™, or ECCOXOLAC™, optionally administered at between about 10 mg to 2000 mg daily, optionally in divided doses),
a sulfonamide (optionally a nimensulide, optionally AULIN™, MESULIDE™, or NIMED™) optionally administered at between about 10 mg to 1000 mg daily),
a salicylate (optionally a acetyl salicylic acid or aspirin, optionally administered at between about 40 mg to 4000 mg daily, optionally in divided doses),
an indole or an indene acetic acid (optionally an indomethacin, optionally INDOCIN™, INDOCID™, INDOCHRON E-R™, or INDOCIN-S™, optionally administered at between about 10 mg to 400 mg daily, optionally in divided doses),
a heteroaryl acetic acid (optionally a diclofenac, optionally CATAFLAM™, or ZIPSOR™), optionally administered at between about 10 mg to 400 mg daily, optionally in divided doses),
an arylpropionic acid, optionally an ibuprofen (optionally BRUFEN™, NUROFEN™, ADVIL™, or NUPRIN™), optionally administered at between about 10 mg to 4000 mg daily, optionally in divided doses:
a naproxen, optionally ALEVE™, ANAPROX™, ANTALGIN™, FEMINAX ULTRA™, FLANAX™, INZA™, MIDOL EXTENDED RELIEF™, MIRANAXV, NALGESIN™, NAPOSIN™, NAPRELAN™, NAPROGESIC™, NAPROSYN™, NAROCIN™, PROXEN™, SYNFLEX™, or XENOBID™, optionally at 10 mg to 4000 mg daily, optionally in divided doses,
a fenamate, optionally a mefanamic acid, optionally PONSTEL™, optionally administered at between about 100 mg to 4000 mg daily, optionally in divided doses,
an enolate (optionally a meloxicam, optionally MOVALIS™, MELOX™, RECOXA™, MOBIC™, MOBEC™, MOBICOX™, TENARON™, ILACOX™, MAVICAM™, MELOCAM™, or ARTRIFLAM™) optionally administered at between about 1 mg to 100 mg daily; optionally administered at between about piroxicam at 1 mg to 100 mg daily),
an alkanone (optionally a nabumetone, optionally RELAFEN™, RELIFEX™, or GAMBARAN™) optionally administered at between about 10 mg to 4000 mg daily, optionally in divided doses), or
any combination thereof (e.g., optionally comprising any non-steroidal anti-inflammatory drug (NSAID), e.g., comprising aspirin, diclofenac; diflunisal, etodolac, fenoprofen, flurbiprofen, ibuprofen, indomethacin, ketoprofen; ketorolac, meclofenamate, mefenamic acid, meloxicam, nabumetone, naproxen, oxaprozin, piroxicam, salsalate, sulindac, tolmetin, a COX-2 inhibitor (e.g., a COX-2-selective inhibitor) or a combination thereof, wherein optionally the COX-2-selective inhibitor comprises celecoxib rofecoxib, etoricoxib, valdecoxib, parecoxib, meloxicam or lumiracoxib).

15. The product of manufacture of claim 1, wherein the compound, composition or formulation comprises or is formulated as:
a tablet, a pill, a lozenge, a capsule, a gel, a geltab, a nanosuspension, a nanoparticle, a microgel and/or a pellet, and optionally the tablet, pill, lozenge, capsule, gel, geltab, nanosuspension, nanoparticle, microgel and/or a pellet are released upon opening of a single package or packet package, or upon opening of a plurality of packages in a blister pack or in a plurality of blister packettes, or
an ampoule, a gel, a lotion, a cream, an emollient, a skin patch or adhesive, aerosol or a spray for topical application, wherein optionally ampoule, a gel, a lotion, a cream, an emollient, a skin patch or adhesive, aerosol or a spray for topical application, and optionally packaged in its own (is contained in a single (one)) tube, ampoule or packette.

16. The product of manufacture of claim 1, further comprising instructions for use; wherein optionally the instructions for using the product of manufacture comprise instructions for use to ameliorate, diminish, treat, block or prevent a systemic inflammation, or an inflammatory response secondary to an infection, a viral infection and/or a reactivation,
and optionally the instructions for using the product of manufacture comprise instructions for use to ameliorate, diminish, treat, block or prevent an inflammation associated with a herpes virus infection, a human herpes virus-8 (HHV-8) infection, or a Kaposi's Sarcoma Herpes Virus infection; or
optionally the instructions for using the product of manufacture comprise instructions for use to ameliorate, diminish, treat, block or prevent an inflammation associated with a hyperproliferative skin disorder, Kaposi's Sarcoma, an inflammation secondary to HIV-induced immunosuppression, a B-cell disorder, Castleman's Disease, or Multicentric Castleman's Disease (MCD).

17. The product of manufacture of claim 1, further comprising a compound, a pharmaceutical composition or a formulation or therapy comprising:
a compound, a pharmaceutical composition or a formulation or therapy comprising:
a compound, a pharmaceutical composition or a formulation or therapy targeting a cell infected with human herpes virus-8 (HHV-8),
a rituximab (optionally RITUXAN™, or MABTHERA™) or other antibodies that target a CD20 antigen expressed on a B-cell, optionally administered at between about 10 mg/m² to 1000 mg/m² given by infusion, up to every two weeks),
an etoposide (optionally EPOSIN™, ETOPOPHOS™, VEPESID™, or VP-16™) or other inhibitors of a eukaryotic topoisomerase II (optionally based on podophyllotoxin), optionally administered at between about 10 mg/m² up to 100 mg/m², optionally taken orally in divided doses or given by intravenous infusion or any other route, or
any combination thereof.

18. The product of manufacture of claim 1, further comprising a compound, a pharmaceutical composition or a formulation or therapy comprising:
a compound, a pharmaceutical composition or a formulation or drug that modulates the immune system, or causes an HHV-8 reactivation due to an alteration in an immune system function,
a thalidomide, a lenalidomide, a pomalidomide, or a congener or analog (optionally THAIXOMID™, or REVLIMID™) optionally administered at between about 10 mg to 1000 mg daily, in divided doses, with or without concomitant glucocorticoid therapy,
a lenalidomide (optionally administered at between about 1 mg to 100 mg daily, with or without concomitant glucocorticoid therapy),
a pomalidomide (optionally administered at between about 0.1 mg to 40 mg daily, or every other day, with or without concomitant glucocorticoid therapy), or
any combination thereof.

19. The product of manufacture of claim 1, further comprising a compound, a pharmaceutical composition or a formulation or therapy comprising:
a compound, a pharmaceutical composition or a formulation or drug that modulates an immune system by interfering with a calcineurin/NFAT signaling in a T cells;
a cyclosporine, a cyclosporine A, or ciclosporin (optionally administered at between about 0.1 mg/kg/da to 20 mg/kg/da),
a tacrolimus (optionally FK-506™ or FUJIMYCIN™) optionally administered at between about 1 μg/kg/da up to 100 μg/kg/da by, optionally intravenous infusion),
a pimecrolimus (optionally ELIDEL™) (optionally administered at between about 1 μg/kg/da up to 100 μg/kg/da, optionally by intravenous infusion), or
any combination thereof.

20. The product of manufacture of claim 1, further comprising a compound, a pharmaceutical composition or a formulation or therapy comprising:
(a) a compound, a pharmaceutical composition or a formulation or drug that acts directly or indirectly as an anti-proliferative agent for a T cell,
a sirolimus or rapamycin (optionally RAPAMUNE™) optionally administered at between about 1 mg up to 100 mg/da, optionally by oral or intravenous route,
an inhibitor of a mammalian target of rapamycin (mTORs),
an azathioprine (optionally administered at between about 0.1 mg/kg/da up to 10 mg/kg/da, optionally by oral, intravenous, or other route),
a mycophenolate mofetil or a mycophenolic acid (optionally CELLCEPT™ or MYFORTIC™) optionally administered at between about 100 mg up to 10 g/da, optionally by oral or intravenous, or
any combination thereof; or
(b) a compound, a pharmaceutical composition or a formulation or therapy comprising:
a compound, a pharmaceutical composition or a formulation or drug that interferes with signaling through an IL-6 cytokine pathway, or attenuates an immunomodulatory response induced by IL-6,
a monoclonal antibody that binds to an IL-6 receptor,
a tocilizumab (optionally ACTEMRA™ or ROACTEMRA™) optionally administered at between about 1 mg/kg up to 20 mg/kg, optionally given by intravenous infusion, optionally as often as every 28 days; optionally a dosing schedule including daily infusions,
a monoclonal antibody that adsorbs an IL-6 peptide,
an elsilimomab (optionally B-E8™) administered at between about 1 mg/kg up to 10 mg/kg every 2 weeks), optionally given with or without concomitant glucocorticoid therapy, or
any combination thereof; or
(c) a compound, a pharmaceutical composition or a formulation or therapy comprising:
a compound, a pharmaceutical composition or a formulation or drug that is a cytotoxic drug,
a compound, a pharmaceutical composition or a formulation or drug that is a cancer chemotherapeutic,
a compound, a pharmaceutical composition or a formulation or drug that induces myelosuppression,
a compound, a pharmaceutical composition or a formulation or drug that is disrupts microtubule function,
a taxane (optionally paclitaxel or TAXOL™, or docetaxel or TAXOTERE™),
a polyether macrolide (optionally halichondrin B, or eribulin or HALAVEN™),
a compound, a pharmaceutical composition or a formulation or drug that inhibits a DNA methyltransferase activity,
an azacytidine, optionally a 5-azacytidine, optionally VIDAZA™, optionally administered at between about 10 mg/m²/da up to 300 mg/m²/da, optionally by subcutaneous injection, or optionally by intravenous infusion,
a decitabine, optionally a DACOGEN™, optionally administered at between about 1 mg/m² up to 100 mg/m² up to three times daily, optionally by intravenous infusion,
a depsipeptide drug that inhibits a histone deacetylase,
a romidepsin, optionally ISTODAX™, optionally administered at between about at 1 mg/m² up to 100 mg/m² weekly, optionally more or less frequently by intravenous infusion, or
any combination thereof
